(19) United States
(12) Patent Application Publication
(10) Pub. No.: US 2012/0288573 A1
(43) Pub. Date: Nov. 15, 2012

(54) USE OF EXTRACTS FROM SALONUM GLAUCOPHYLLUM FOR TREATING BONE METABOLISM DISORDERS AND KIDNEY DISORDERS

(75) Inventors: Heinrich Bachmann, Wintersingen (CH); Sabrina Autzen, Reutlingen (DE)

(73) Assignee: HERBONIS AG, Basle (CH)

(21) Appl. No.: 13/502,922

(22) PCT Filed: Oct. 20, 2010
(86) PCT No.: PCT/EP2010/065784

§ 371 Date: May 23, 2012

(30) Foreign Application Priority Data
Oct. 20, 2009 (EP) PCT/EP2009/007518

(51) Int. Cl. A61K 31/7048; A61P 3/00; A61K 38/17; A61P 3/02; A61K 33/06; A61K 33/10; A61P 19/08; A61P 13/12

(52) U.S. Cl. 424/686; 514/27; 424/682; 514/5.7

(57) ABSTRACT

A method is provided for preventing or treating bone or musculoskeletal metabolism disorders or kidney malfunction in humans. It comprises providing to a subject in need of that a calcium containing or calcium enriched diet combined with the administration of an efficient amount of a plant extract of Solatium glaucophyllum comprising from about 0.8 to about 2.2 weight % of a mixture of 1,25-dihydroxyvitamin D3 glycosides and quercetin glycosides present in a ratio of about 1:100 to 1:200 parts by weight. There are also provided food or dietary composition and food supplement designed for preventing or treating bone or musculoskeletal metabolism disorders or kidney malfunction in humans which comprises an efficient amount of the said plant extract of Solanum glaucophyllum and a source of calcium.

### FIELD OF THE INVENTION

The invention refers to the field of nutrition and more specifically to a nutritional approach of the prevention or treatment of specific, middle to severe bone or musculoskeletal metabolic troubles in humans.

### BACKGROUND OF THE INVENTION

During the last decades the average age and the life expectancy of the population, among in particular the western population, have increased constantly so that people being over 65, respectively over 75 or even 85 years old represent now a significant proportion of the global population. Experts are awaiting an increase of up to 20% of that population segment for the next two decades in western countries what shall lead to a situation which will obviously generate a parallel increase of related social or health concerns.

Concerning the evolution of the health status of this specific segment of the population one has already noticed a drastic increase of physical or mental troubles or disorders like e.g. diabetes, Alzheimer disease or senility related troubles like mild to sever depressive mood, impaired mental or cognitive or memorization capacity and now more and more frequently bone metabolism deficiencies like e.g. osteopenia, senile osteoporosis or postmenopausal osteoporosis.

Many approaches have been suggested and are pursued to prevent age-related and postmenopausal bone mass reduction. As of today, known anti-osteoporotic drugs include e.g. bisphosphonate compounds, synthetic estrogens, Vitamin D, Vitamin D derivatives and Vitamin D metabolites.

Concerning the administration of Vitamin to patients is known that Vitamin D3 is progressively transformed in situ into active metabolites which can control to some extent calcium uptake in the intestinal tract and its deposition into and mobilization from bone.

A negative drawback, however, of the administration of Vitamin D3 metabolites and its synthetic analogues is the particularly narrow therapeutic window for medication and the risk of hypercalcemia, i.e. an abnormally and toxic high blood concentration of calcium.

As to whether this therapy is suitable nonetheless depends on further factors, e.g. the costs for preparing the active compounds used therein. The bottle neck for a cost efficient therapy of diseases as mentioned above using Vitamin D3 metabolites or its synthetic analogues is consequently a cheap provision or preparation of Vitamin D metabolites.

PCT/EP2008/003191 to Herbonis AG has recently proposed a solution designed to overcome most of if not all the drawbacks or negative side effects of the administration of active Vitamin D3 in the treatment of bone disorders like e.g. postmenopausal osteoporosis.

This document refers to a method for preparing and purifying a plant extract of Solanum glaucophyllum having an enriched content of specific Vitamin D3 metabolites, namely 1,25-dihydroxy-vitamin-D3 glycosides—wherein 1,25 -dihydroxy-vitamin-D3 -1β-glucopyranoside one of their representatives—and quercetin glycosides: in this context the term “enriched” applies to highly purified plant extracts comprising at least 300 ppm, preferably from min. 500 up to 2000 ppm of the said Vitamin D3 metabolites and at least 10%, preferably min. 15% up to 20% of quercetin glycosides.

Such enriched and highly purified plant extracts are described as suitable for the preparation of pharmaceutical compositions useful in the treatment of bone mass reduction diseases in humans and animals also but, so far, doctors or vets are not yet able to prescribe the administration of these natural active ingredients to humans either for preventing or for treating the targeted bone mass reduction diseases.

Typically senile osteoporosis represents today an increased medical and social concern for the medical community especially when the latter have to implement prevention thereof in the aged population: as additional physical or mental troubles appear frequently simultaneously it is extremely hard to rely on the concerned persons for a self medication. Dosage is consequently often inadequate; the recommended daily administration is often not followed at all and, eventually, the whole health status is increasingly impaired.

Here also the primarily bottleneck is the achievement of an adequate cost controlled and efficient prevention or treatment and this is still more true when contemplating “mild” troubles like osteopenia or osteoporosis in said high aged population.

The present invention has the merit to offer to the community a very convenient, cheap and efficient way of administration of highly performing active ingredients like Vitamin D3 metabolites to a widely spread population facing health troubles like e.g. senile osteoporosis.

The invention provides a “nutritional approach”, as opposed to a therapeutic approach, to the above captioned problem and more concretely an original solution in the form of dedicated food or dietary compositions, dedicated food supplements and related methods of prevention or treatment as well. The invention enables indeed an easy set up, day after day, of a way of administration of active ingredients which is entirely compatible with the usual way of life of the concerned people, whether they are lightly or strongly affected by the troubles linked to senility.

The invention is defined in the claims provided here below.

### SUMMARY OF THE INVENTION

The invention provides first a method for preventing or treating bone or musculoskeletal metabolism disorders or kidney malfunction in humans which comprises providing to a subject in need of that a calcium containing or calcium enriched diet combined with the administration of an efficient amount of a plant extract of Solanum glaucophyllum comprising from about 0.8 to about 2.2 weight % of the natural mixture of 1,25-dihydroxyvitamin D3 glycosides and quercetin glycosides present in a ratio of about 1:100 to 1:200 parts by weight.

The invention further provides the use of a plant extract of Solanum glaucophyllum comprising from about 0.8 to about 2.2 weight % of a mixture of 1,25-dihydroxyvitamin D3 glycosides and quercetin glycosides present in a ratio of about 1:100 to 1:200 parts by weight in the preparation of a dietary or food composition useful for preventing or treating bone or musculoskeletal metabolism disorders or kidney malfunction in humans.

The invention still further provides a food or dietary composition designed for preventing or treating bone or musculoskeletal metabolism disorders or kidney malfunction in humans which comprises an efficient amount of a plant extract of Solanum glaucophyllum comprising from about 0.8 to about 2.2 weight % of a mixture of 1,25-dihydroxyvitamin D3 glycosides and quercetin glycosides in a ratio of about 1:100 to 1:200 parts by weight and a source of calcium.

The invention provides eventually a food supplement designed for preventing or treating bone or musculoskeletal metabolism disorders or kidney malfunction in humans which comprises an efficient amount of a plant extract of Solanum glaucophyllum comprising from about 0.8 to about 2.2 weight % a mixture of 1,25-dihydroxyvitamin D3 glycosides and quercetin glycosides in a ratio of about 1:100 to 1:200 parts by weight and optionally a source of calcium.

### DETAILED DESCRIPTION OF THE INVENTION

The plant extract which is referred to according to the invention is prepared from dry leaves of Solanum glaucophyllum plants by means of a duly dedicated method which comprises, as initial step, maceration or percolation of the raw material with a specifically selected mixture of water and an organic solvent. The resulting extract is subsequently mixed with protective and buffering additives, dried down to predefined dry matter content, optionally sterilized and subsequently passed through an ion exchange column.

The related effluent is eventually filtered and dried or spray dried using moderate temperature conditions; during that later step the resulting plant extract is eventually standardized to the desired content in active ingredients by addition of an inert excipient, e.g. by means of the addition of GRAS ballast material.

Such a plant extract is indeed very soluble in water as well as in most of the liquids usually present in food products. Surprisingly, the said plant extract is almost odorless and exhibits a malt or caramel, slightly cereal like taste and flavor which matches well with most of the food or dietary products contemplated within the frame of the invention.

Last but not least the said extract has proved sufficiently stable when conditioned or processed together with most of the usual food ingredients—see stability trials referred to below—for example when preparing spray dried soluble powders, preparing cereal flakes using twin screw extrusion or mixing with fruit or vegetable juices.

As confirmed by the pharmacokinetics studies referred to here below the said Solanum glaucophyllum plant extract has also the advantage to deliver its active ingredients, and namely 1,25-dihydroxyvitamin D3 glycosides, in a particularly efficient way when considering their bioavailability. In particular, these experiments have demonstrated the effectiveness of the administration of Solanum glaucophyllum extract according to the invention when leading to a dose dependent increase of blood calcium and phosphorus, i.e. a necessary condition for prevention osteoporosis.

These experiments have further demonstrated that the application of the aforementioned extract of Solanum glaucophyllum is able to supply efficiently the individual with 1,25-dihydroxyvitamin D₃, i.e. the active metabolites of vitamin D₃.

Kidney is in fact the only body tissue which would convert vitamin D into its active form, i.e. 1,25-dihydroxyvitamin D₃. Consequently, in patients affected by an impaired kidney function this metabolic transformation cannot take place: Solanum glaucophyllum extract is in fact the source of active vitamin D3 metabolites which would enable to bypass this deficiency efficiently.

According to the invention Solanum glaucophyllum plant extracts comprise from about 0.8 to about 2.2, e.g. 1.0 or 1.5, 2.0 or 2.2 weight % of a mixture of 1,25-dihydroxyvitamin D3 glycosides and quercetin which represent there the key active ingredients. More important is the control of the specific ratio of these ingredients which are present in a ratio of about 1:100 to 1:200, e.g. 1:100, 1:120, 1:140, 1:150, 1:160, 1:180 or 1:200. Monitoring and controlling both the concentration and the ratio of the said active ingredients in the final plant extract is crucial.

Such a plant extract can be prepared from any wild or cultivated variety provided the latter exhibits a minimum content of the above active ingredients. A particularly suitable variety is represented by Solanum glaucophyllum Desf., bearing the designation “HERVIT” according to Community Plant Variety Certificate EU 25473 of Jun. 22, 2009 as it provides the highest concentration achievable today.

The method of preparation briefly disclosed here above allows obtaining a plant extract free of alkaloids genuine to Solanum glaucophyllum or, expressed differently, definitely below the detection level, so the said extract does not or cannot be suspected to exhibit negative side effects due to excessive or uncontrolled amounts of said alkaloids.

Quercetin glycosides are also present in the said plant extract and they act in situ in combination with the formerly mentioned 1,25-dihydroxyvitamin D3 glycosides; they comprise essentially rutin, apiosyl rutin, hyperoside, isoquercitrin and other minor quercetin oligoglycosides.

The invention is providing a method for preventing or treating bone or musculoskeletal metabolism disorders or correcting kidney malfunction in humans which comprises providing to a subject in need of that: as bone metabolism disorders the invention contemplates, among others, osteopenia, post menopausal osteoporosis, senile osteoporosis, suboptimal bone development during fast bone growth periods and bone mass reduction due to extreme sport efforts.

Treating or preventing musculoskeletal disorders means essentially promoting or maintaining musculoskeletal mass or function.

Concerning treatment or prevention of kidney malfunction in humans the invention contemplates mainly that of vitamin D resistant rickets and renal insufficiency, by enabling delivering the body with the essential active vitamin D₃ metabolite. This represents a crucial advantage because a damaged kidney cannot produce the essential vitamin D3 metabolite from the precursor circulating in blood plasma.

The invention further provides to the persons in need, together with the said plant extract, a calcium containing or calcium enriched diet, in fact any convenient solid or liquid food product containing a predefined amount of a source of edible (bioavailable) calcium, preferably selected from edible organic or inorganic calcium salts, organic or inorganic edible calcium complexes and in particular calcium carbonate, magnesium calcium carbonate in particular activated dolomite, calcium caseinate, calcium glucuronate, calcium lactate, calcium citrate or casein.

The term “diet” means here a complete daily diet proposed to the subject, e.g. a person affected by senile osteoporosis and which provides any necessary nutrients like carbohydrates, fats, proteins, minerals and water, of course adjusted to the health status of the said person.

Good results have been observed in respect of a diet providing over one day (e.g. 3 to four meals) an amount of mixture of 1,25-dihydroxyvitamin D3 glycosides and quercetin glycosides, in the form of Solanum glaucophyllum plant extract as referred to here above, comprised between about 5 to 200 mg, preferably of about 10 to 100 mg for a subject weighing approximately 65 Kg.

The amount of calcium provided daily, simultaneously with the said plant extract is generally of about 400 to about 1600 mg, preferably of about 400 to 1200 mg for a subject approximately about 65 Kg and is of course depending on the amounts of calcium already present in the food product in consideration as well as on the type of the source of calcium selected therefore.

Depending on the subject or the type of meal in consideration the Solanum glaucophyllum plant extract can be administered either simultaneously, i.e. during the meal and preferably in the most attractive portion of the meal or sequentially, in that case preferably as food supplement (see below).

According to the invention almost any food or dietary composition is suitable for achieving the desired effect provided its is adapted to the age, the regime or the health status of the person involved and may comprise hot liquid foods like soups or bouillons, hot or chilled beverages like milk, fruit juice. As suitable solids or semi solid food products one may consider mashed vegetables, dairy food products like yogurts, fresh cheese, flans or puddings; cereal base products like cereal bars or cereal flakes.

Such a wide choice offers the possibility to propose along the day, the week or during the whole period of treatment an almost unlimited range of meals which shall remain attractive to the concerned person and which, consequently, shall guarantee day after day the adequate intake or administration of the active ingredients referred to here above.

Consequently, the invention has also as an object any food or dietary composition added with Solanum glaucophyllum plant extract comprising from about 0.8 to about 2.2 weight % of a mixture of 1,25-dihydroxyvitamin D3 glycosides and quercetin glycosides present in a ratio of about 1:100 to 1:200 parts by weight and fulfilling the conditions highlighted here above.

Next to said food or dietary compositions the invention has as further object a food supplement designed for preventing or treating bone or musculoskeletal metabolism disorders or kidney malfunction in humans comprising an efficient amount of the plant extract of Solanum glaucophyllum mentioned in the above context.

The said food supplement can be an edible, e.g. starch based or dextrose or dextrin or cellulose based powder comprising a predefined amount of Solanum glaucophyllum comprising from about 0.8 to about 2.2 weight % of a mixture of 1,25-dihydroxyvitamin D3 glycosides and quercetin glycosides present in a ratio of about 1:100 to 1:200 parts by weight.

The latter can be stored as such and merely added to the convenient food or beverage prior eating: alternatively the food supplement in powder form can be used for the preparation of tablets and, preferably, edible capsules which are then proposed with a separate beverage.

The examples provided below simply constitute an illustration of some of the numerous and diversified food products or food supplements which can be used according to the invention.

### EXAMPLE 1

### Preparation of a Solanum glaucophyllum Plant Extract

a) Maceration Variant

A selected batch of dry leaves of Solanum glaucophyllum has been macerated under agitation at 40- 60° C. for about 24 hours with the 5 to 12 liter per kg of a water/ethanol mixture.

Previous analytical assessment of the content in 1,25-dihydroxyvitamin D3 glycosides of various batches of dry leaves material available in the storehouse led to prepare an 85/15** in volume water/ethanol mixture. The use of the proper mixture allows the achievement of the desired content of vitamin D3 derivative—approx. 100 ppm in this example—as well as the ratio of 1,25-dihydroxyvitamin D₃ glycosides and quercetin glycosides. The liquid fraction was separated and set aside whereas maceration was repeated for a second turn of 24 hours.

(**similar results have been achieved using mixtures comprising ca 20 to 40% ethanol)

The collected water/ethanol extracts were then combined and added with ascorbic acid in a ratio of 0.25% by weight and pH of same was adjusted to 5-6.5 by means of a food-compatible acid, citric acid in this particular case.

The stabilized extract was then filtered and concentrated to approx. 30 to 50% of dry matter content by means of vacuum drying technique and the resulting concentrated was poured onto a cation exchanger resin equilibrated in its H⁺ form.

The collected effluent was filtered and then subjected to UHT sterilization (125° C. for 3 s). After analytically assessment of the 1,25-dihydroxyvitamin D₃-glycosides and quercetin-glycosides content, a specific amount of excipient, preferable maltodextrin, lactose or corn starch, is added the sterilized material in order to standardize the final content of active ingredients in the plant extract—100 ppm of 1,25-dihydroxyvitamin D3.

The standardized mixture is eventually spray dried or vacuum dried in a conventional spray drier to afford the desired Solanum glaucophyllum plant extract in powder form.

b) Percolation Variant

A selected batch of dry leaves of Solanum glaucophyllum—see above—has been subjected to percolation at 35 to 45° C. by means of an 85/15** in volume mixture of water and ethanol. The percolation took place in conventional equipment using 4 to 6 vessels in a cyclical regime. A solvent ratio of 8 to 12, e.g. 10 parts by weight for 1 part of dry leaves was applied and percolation was pursued over 2 to 4 hours.

(**similar results have been achieved using mixtures comprising ca 20 to 40% ethanol)

The percolated extract was the subjected to the same subsequent steps as for the maceration variant in order to achieve a final plant extract comprising ca. 100 ppm of 1,25-dihydroxyvitamin D3 and quercetin glycosides in a ratio similar to that afforded according to the above maceration step.

### EXAMPLE 2

### Calcium Enriched Milk

Calcium enriched liquid milk was prepared by mixing at room temperature 10 liters of tap water with 1200 g of partially defatted milk powder and 40 g of calcium magnesium carbonate (activated dolomite). Then 20 g of the plant extract of Example 1 have been progressively poured into the reconstituted milk and the resulting liquid was distributed in portions of ca. 250 ml each and eventually stored in a fridge.

Each reconstituted milk portion comprises ca. 500 mg of plant extract which represents the half of the adequate daily dose recommended for a person suffering or suspected to suffer of senile osteoporosis, osteopenia or for post menopausal women.

It has been observed that the reconstituted milk was well accepted in terms of taste and flavor. Depending on the personal taste of the person sugar, cocoa powder of soluble coffee powder can be added thereto; also the said milk or milk based beverage can be heated on request.

### EXAMPLE 3

### Calcium Enriched Orange Juice

Commercially available orange juice (1 L) was added under stirring with 4 g of activated dolomite and 0.25 g of the plant extract of Example 1 and eventually stored in the fridge.

A portion of ca. 250 ml of said orange juice is providing the adequate daily dose of plant extract suitable for a person suffering or suspected to suffer of senile osteoporosis, osteopenia or for post menopausal women.

The latter is recommended for the breakfast or at lunch; the typical orange taste and flavor are not affected by the addition of these two ingredients.

### EXAMPLE 4

### Bouillon Powder

Commercially available bouillon in dehydrated powder form (1 Kg) was mixed with 50 g of plant extract according to Example 1.

Pouring 150 ml of hot water at around 80° C. onto 2 g of the above powder provides a clear bouillon suitable for preparing a lunch which can be competed by Calcium containing food like e.g. cheese, fresh cheese, yogurt or a milk based dessert cream. Such a meal can be proposed to a person suffering or suspected to suffer of senile osteoporosis, osteopenia or to post menopausal women.

### EXAMPLE 5

### Calcium Containing Cereal Bar

A composition for preparing extruded cereal bars can be obtained by means of the following ingredients

| Ingredients | Parts by weight (g) |
|---|---|
| High fructose corn syrup | 400 |
| Oat/Wheat bran | 100 |
| Maltodextrin | 100 |
| Skimmed milk powder | 100 |
| Rice flour | 50 |
| Cereal crisp | 150 |
| Commercial vitamin premix | 10 |
| Commercial almond paste | 20 |
| Commercial mineral premix | 10 |
| Commercial vanilla flavor | 10 |
| Plant extract of Example 1 | 5 |

Each of cereal bar resulting of a standard extrusion process weighs 10 g and provides ca. 50 mg of the plant extract of Example 1 to the consumer. Depending on the needs (sports men) or the health status of the concerned person (e.g. senile or menopausal status) a daily dose may comprise from one to three or even four cereal bars.

### EXAMPLE 6

### Cereal Flakes for Breakfast

A composition for preparing extruded cereal flakes can be obtained by means of the following ingredients:
Wheat flour (whole grain flour)
Rice flour
Corn syrup
Barley malt extract
Calcium carbonate
Commercial mineral mix (incl. buffers)
Commercial vitamin mix (inc. antioxidants)

Addition of predefined amounts of the plant extract of Example 1 can be achieved according to distinct ways: the said extract is first dissolved in sucrose syrup and the latter is added to the above composition which is eventually subjected to twin screw extrusion and subsequent drying to afford calcium containing or, depending on the amount added, calcium enriched cereal flakes.

Alternatively, the said extract is first dissolved in sucrose syrup and the latter is eventually sprayed onto the cereal flakes as extruded and before drying.

In both cases it was observed that the typical “breakfast cereal flake” taste and flavour were not affected by the addition of the said plant extract.

Depending on the specific regime or diet of the concerned person one can propose a breakfast menu comprising milk or yogurt or fresh cheese to eat together with the above cereal flakes.

### EXAMPLE 7

### Calcium Containing Food Supplement

Edible cellulose capsules (hydroxypropyl methyl cellulose) each comprising 50 mg of the plant extract of Example 1 have been manufactured using filler comprising the following ingredients:
Dehydrated yogurt powder
Anhydrous dextrose
Potato starch
Microcrystalline cellulose
Plant extract of Example 1

Depending on the needs (e.g. sports men) or the health status of the concerned person (e.g. senile status, menopausal status) a daily dose may comprise from one to three or even four edible capsules.

### EXAMPLE 8

### Stability Trials in Food Products or Beverages

An aliquot of Solanum glaucophyllum extract according to Example 1 was added with stirring to various commercially available liquids or food products at room temperature or upon heating in a few cases, then stored at ca 4° C., respectively ca 37° C. concerning the powders, over a prolonged period. Samples were taken there from periodically (Day 0, Day 7, etc. . . .) and, eventually, subjected to a quantitative determination of their content in 1,25-dihydroxyvitamin D3 glycosides.

The Tables below are summarizing the corresponding analytical results, which are expressed in % of the amount of Solanum glaucophyllum extract (Solbone) added initially (+/−15%).

| Food/Beverage | Day 0 | Day 4* | Day 7* | Day 21* | Day 35* |
|---|---|---|---|---|---|
| Orange juice | 100 | 97 | 90 | 101 | 93 |
| Milk | 100 | 106 | 114 | 72 | 71 |
| Water | 100 | 88 | 96 | 129 | 107 |
| Isotonic drink | 100 | 104 | 121 | 97 | 104 |
| Ice tea | 100 | 113 | 78 | 97 | 93 |
| Yogurt | 100 | 87 | 88 | 79 | 89 |
| Hot water** | 100 | 104 | 84 | 84 | 108 |
| Hot tea** | 100 | 100 | 133 | n.a. | 94 |

*storage temperature: +4° C.

| Beverage | | | | | |
|---|---|---|---|---|---|
| powder | Day 0 | Day 4** | Day 7** | Day 21** | Day 35* |
| Nesquik ® | 100 | 89 | 96 | 94 | n.a. |
| Icetea | 100 | n.a. | 109 | 65 | 52 |
| Beef bouillon | 100 | 94 | 96 | 116 | 98 |

**storage temperature: +37° C.

These tables illustrate to which extent the tested Solanum glaucophyllum extract is stable in the most frequently met food products or beverages.

### EXAMPLE 9

### Pharmacokinetic Studies in Larger Mammalian Animals

### 9.1 Application in Goats

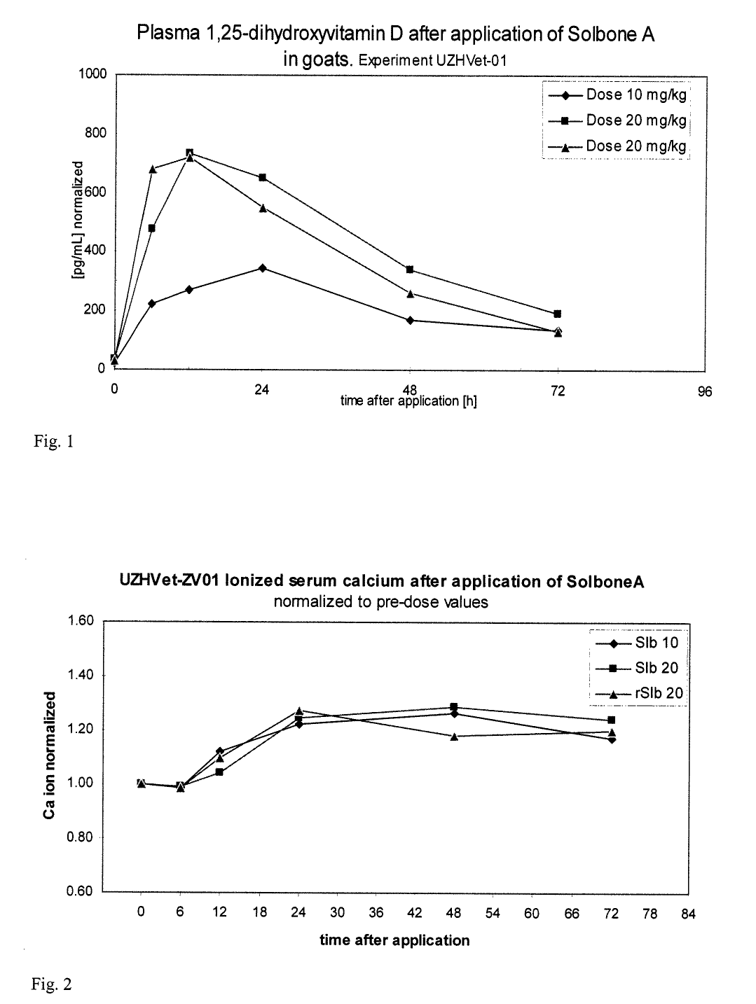

A single dose of Solanum glaucophyllum extract according to Example 1 (identified as Solbone®) representing 10, respectively 20 mg per kg body weight was applied to 4 goats. Blood samples which were collected before and, periodically, after the application over an overall period of 96 hours were then subjected to a quantitative analysis of 1,25-dihydroxyvitamin D3 (referred as 1,25(OH)₂D₃). The corresponding results are reported in FIG. 1 which exhibits the plasma distribution of 1,25(OH)₂D₃ over the above mentioned period.

Serum calcium measurements were also performed in parallel using the blood samples referred to here above. The corresponding results are reported in FIG. 2 and illustrate the effect of the extract on the calcium distribution in blood plasma.

9.2 Application in Dairy Cattle

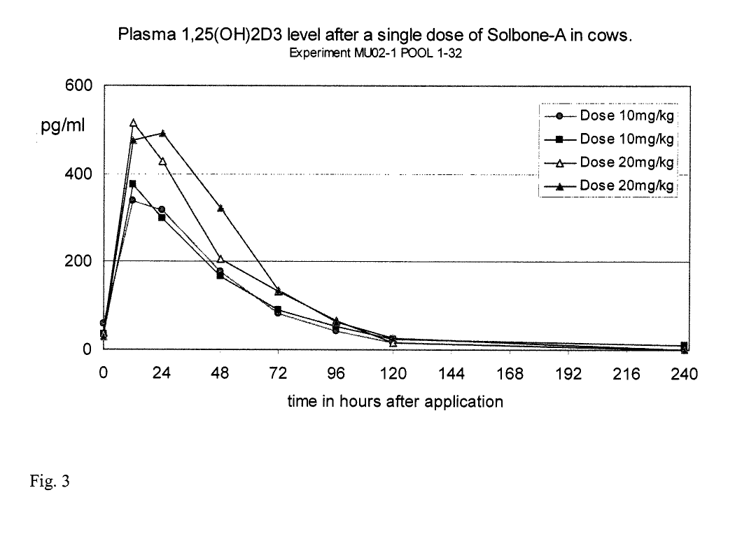

A single dose of Solanum glaucophyllum extract according to Example 1 (identified as Solbone®) representing 10, respectively 20 mg per kg body weight was applied to 5 cows each treatment group. Blood samples which were collected before and, periodically, after the application over an overall ten days period were then subjected to a quantitative analysis of 1,25(OH)₂D₃. FIG. 3 provides exhibits the dose dependent increase of 1,25(OH)₂D₃ and its exhaustion over the above mentioned period.

The table below shows the serum calcium and phosphate levels in this experiment and demonstrates a significant increase of the minerals in blood due to treatment with the Solanum glaucophyllum extract. Furthermore, this experiment also demonstrates that the inventive extract of Solanum glaucophyllum is able to deliver the body with the essential vitamin D metabolite when the kidney fails to produce sufficient amounts of the active form of vitamin D in kidney diseases or in decreasing endogen production due to the age—see also FIG. 3.

| | Serum Calcium (mmol/l) | | Serum phosphor (mmol/l) | | Serum 1.25(OH)₂D₃ (pg/mL) | |
|---|---|---|---|---|---|---|
| day | control | Solbone | control | Solbone | control | Solbone |
| 0 | 2.40 ± 0.06 | 2.50 ± 0.06 | 2.24 ± 0.17 | 2.44 ± 0.06 e | 30 | 30 |
| 1 | 2.44 ± 0.09 | 2.67 ± 0.03 E | 2.07 ± 0.30 | 2.84 ± 0.18 E | 10 | 291 E |
| 2 | 2.39 ± 0.02 | 2.74 ± 0.07 E | 2.12 ± 0.21 | 3.25 ± 0.21 E | 30 | 156 E |
| 3 | 2.42 ± 0.03 | 2.75 ± 0.11 E | 2.22 ± 0.18 | 3.41 ± 0.23 E | 36 | 58 |
| 4 | 2.45 ± 0.05 | 2.72 ± 0.06 E | 2.38 ± 0.22 | 3.45 ± 0.53 E | 8 | 28 |
| 5 | 2.46 ± 0.03 | 2.64 ± 0.16 e | 2.05 ± 0.15 | 2.71 ± 0.41 e | 28 | 53 |
| 7 | 2.44 ± 0.06 | 2.60 ± 0.11 | 2.06 ± 0.10 | 2.66 ± 0.35 E | 19 | 34 |
| 9 | 2.46 ± 0.02 | 2.59 ± 0.07 E | 1.97 ± 0.08 | 2.10 ± 0.16 | 20 | 22 |
| 11 | 2.40 ± 0.05 | 2.44 ± 0.06 | 2.10 ± 0.06 | 2.26 ± 0.34 | 21 | 16 |

E (p < 0.01)
e (p < 0.05)
Solbone vs. control

1. A method for preventing or treating bone or musculoskeletal metabolism disorders or kidney malfunction in humans comprising providing to a subject in need of same a calcium containing or calcium enriched diet combined with a therapeutically-effective amount of a plant extract of Solanum glaucophyllum comprising from about 0.8 to about 2.2 weight % of a mixture of 1,25-dihydroxyvitamin D3 glycosides and quercetin glycosides present in a ratio of about 1:100 to 1:200 parts by weight.

2. The method of claim 1 wherein the bone metabolism disorder is selected from the group consisting of osteopenia, post menopausal osteoporosis, senile osteoporosis, suboptimal bone development during fast bone growth periods and bone mass reduction due to extreme sport efforts.

3. The method of claim 1 which comprises promoting or maintaining musculoskeletal mass or function.

4. The method of claim 1 wherein the kidney malfunction is selected from the group consisting of vitamin D resistant rickets and renal insufficiency.

5. The method of claim 1 wherein the Solanum glaucophyllum is selected from the group consisting of any wild or cultivated variety.

6. The method of claim 1 wherein the Solanum glaucophyllum plant extract is free of alkaloids genuine to Solanum glaucophyllum.

7. The method of claim 1 wherein the quercetin glycosides comprise rutin, apiosyl rutin, hyperoside, isoquercitrin and other minor quercetin oligoglycosides.

8. The method of claim 1 wherein calcium is provided from a source selected from the group consisting of edible organic, inorganic calcium salts, organic and inorganic edible calcium complexes.

9. The method of claim 1 wherein the calcium containing or calcium enriched diet is a daily diet providing necessary nutrients and the daily dose of the mixture of 1,25-dihydroxyvitamin D3 glycosides and quercetin glycosides provided to the subject is of about 5 to 200 mg for a subject weighing approximately 65 Kg.

10. The method of claim 1 wherein the calcium containing or calcium enriched diet is a daily diet providing necessary nutrient to the subject and daily dose of calcium provided to the subject is of about 400 to about 1600 mg, for a subject approximately about 65 Kg.

11. The method of claim 1 wherein a plant extract of Solanum glaucophyllum comprising the mixture of 1,25-dihydroxyvitamin D3 glycosides and quercetin glycosides is administered to the subject simultaneously with the diet or sequentially.

12. A method for preventing or treating bone or musculoskeletal metabolism disorders or kidney malfunction in humans comprising the step of administering a composition comprising a plant extract of Solanum glaucophyllum comprising from about 0.8 to about 2.2 weight % of a mixture of 1,25-dihydroxyvitamin D3 glycosides and quercetin glycosides present in a ratio of about 1:100 to 1:200 parts by weight to an individual in need of same.

13. Food or dietary composition comprising a plant extract of Solanum glaucophyllum comprising from about 0.8 to about 2.2 weight % of a mixture of 1,25-dihydroxyvitamin D3 glycosides and quercetin glycosides in a ratio of about 1:100 to 1:200 parts by weight and a source of calcium.

14-16. (canceled)

17. The food or dietary composition of claim 13 wherein Solanum glaucophyllum is selected from the group consisting of any wild or cultivated variety.

18. The food or dietary composition of claim 13 wherein Solanum glaucophyllum plant extract is free of alkaloids genuine to Solanum glaucophyllum.

19. The food or dietary composition of claim 13 wherein quercetin glycosides comprise rutin, apiosyl rutin, hyperoside, quercetin, isoquercitrin and other minor quercetin oligoglycosides.

20. The food or dietary composition of claim 13 which further provides to the subject any necessary nutrients.

21. The food or dietary composition of claim 13 which is in a form suitable for enteral or parenteral administration.

22. The food or dietary composition of claim 13 wherein the dose of mixture of 1,25-dihydroxyvitamin D3 glycosides and quercetin glycosides which is provided daily to the subject is of about 5 to 200 mg for a subject weighing approximately 65 Kg.

23. The food or dietary composition of claim 13 wherein the dose of calcium provided daily to the subject is of about 400 to 1600 mg for a subject weighing approximately 65 Kg.

24. The food or dietary composition of claim 13 wherein the source of calcium is selected from the group consisting of edible organic and inorganic calcium salts, organic and inorganic edible calcium complexes, magnesium calcium carbonate, calcium caseinate, calcium glucuronate, calcium lactate, calcium citrate and casein.

25-33. (canceled)

34. The food composition of claim 13 wherein the composition is a food supplement.
